(19) United States
(12) Patent Application Publication
(10) Pub. No.: US 2015/0272986 A1
(43) Pub. Date: Oct. 1, 2015

(54) COMPOSITION FOR ALLEVIATING CONDITIONS ASSOCIATED WITH VAGINAL DRYNESS

(71) Applicant: INOFLEX AB, Stockamollan (SE)

(72) Inventor: Stern Leif Einar, Stockamollan (SE)

(21) Appl. No.: 14/434,526

(22) PCT Filed: Oct. 11, 2013
(86) PCT No.: PCT/EP2013/071307

§ 371 Date: Apr. 9, 2015

(30) Foreign Application Priority Data
Oct. 12, 2012 (SE) 1251159-8

(51) Int. Cl. A61K 31/77

(52) U.S. Cl. CPC A61K 31/77

(57) ABSTRACT

A composition for alleviating conditions associated with vaginal dryness comprising a thickener, a non-ionic, water-soluble poly(ethylene oxide) polymer having molecular weight of at least 100,000 Da, and at least 95 wt % water.

### FIELD OF THE INVENTION

The present invention relates to a composition for the alleviating conditions, e.g. pain, irritation, inflammation, and/or itching, associated with vaginal dryness, such as vaginal dryness due to decreased estrogen production, vaginal dryness due to medication, and vaginism or vestibulitis.

### BACKGROUND

As in all mucosal orifices of the human body, the vaginal mucosa and vaginal introitus is protected by a thin film of mucus. The mucus film has two main functions:

1) to protect the upper genital tract from infection; and

2) to separate the vaginal walls and to lubricate and protect from dryness, but also to prevent pain during sexual intercourse.

Plasma seepage from vaginal walls due to vascular engorgement is considered to be the main lubrication source, but lubrication is also produced by glands in the vaginal introitus/Bartholinis glands/ as well as by the cylinder epithelium of the cervix. The vagina itself has no mucus glands.

The lubricating fluid usually has a pH of 3.2 to 4.5. The low pH assists in protecting the upper genitals from major pathogens. For instance, the risk of transmission of HIV, and other sexually transmitted diseases (STD), is increased by lacerations of the vaginal tissue due to lack of lubrication, facilitating blood-to-blood contact. The lubricating fluid may vary in consistency, texture, taste, color, and odor, depending on, the phase in the menstrual cycle infection, drugs, genetic factors, sexual arousal and diet. However, vaginal lubrication or moistness is normally always present. The production of mucus increases during sexual arousal and around the time for ovulation, but may also increase during stress and in vaginal infections. Infected vaginal fluor, however, has totally different characteristics from normal lubrication.

Insufficient lubrication or vaginal dryness often results in irritation and a stinging and burning pain in the intoitus and lower vagina. Further, itching is typically also experienced by women suffering from vaginal dryness. Vaginal dryness is highly uncomfortable in everyday life for many women.

The most common reason for insufficient lubrication is the decrease in estrogen production starting in the mid forties, through the menopause and increasing with age. Decrease in estrogen results in depletion of glucogen in the vaginal epithelium with atrophia and epithel vulnerability to trauma and infections.

Iatrogenic atrophy may be produced by anti-estrogenic medication in women operated because of estrogen dependent tumors as breast tumors, some types of ovarian tumors and tumors in the uterine endometrium. Certain other medications, including over-the-counter anti-histamines and medications with anti-cholinergic or sympathomimetic effects will inhibit lubrication as will some drugs for allergic, cardiovascular, psychiatric, and some other medical conditions.

Further, oral contraceptives may decrease vaginal lubrication, as contraceptive creams and foams also may. Even fear or anxiety about sexual intimacy, may decrease vaginal lubrication.

Decreased vaginal lubrication or vaginal dryness may cause pain, irritation and itching in the vaginal introitus and the lower vagina. A special type of vaginal problem sometimes associated with itching is pregnancy and/or breast-feeding (athrophia paradoxa). Vaginism/Vestibulitis—an idiopathic state with pain and cramps in the vagina at penetration which is—at least partly—caused by insufficient lubrication.

Existing treatment of decreased vaginal lubrication or vaginal dryness includes two major categories of treatments:
Hormones—preferably estriol; and
Gels—preferably oil- or silicone-based.

Estriol is the most common hormone used for women with mucosal atrophy from hormone deficit. It may be supplied as tablets, cream or suppositories. However, hormones cannot be used by patients with hormone dependent tumors. Further, many women are afraid of hormone intake. Especially, as hormone intake has been associated with increased risk of cancer. It is generally accepted that estrogens may increase the risk of introducing hormone dependent tumors in the breast, as well as in the endometrium. Some ovarian tumors may also be estrogen dependent. Tablets and suppositories containing hormones do not provide a local mechanical sliding effect, but are acting through systemic effects.

In treating decreased vaginal lubrication or vaginal dryness, hormones may be supplemented by or replaced with oil, silicone or water based gels to support the insufficient inherent vaginal lubrication. Gels of the art may provide sufficient lubrication for sexual intercourse. Further, gels of the art also provide at least some temporarily alleviation of symptoms associated with vaginal dryness, e.g. irritation and itching.

However, gels of the art are often insufficient in providing long term alleviation of symptoms associated with decreased vaginal lubrication or vaginal dryness. Further, gels of the art have no or limited pain soothing effect. Especially, women suffering from vestibulitis do experience no, or very limited, symptom relief from usage of conventional gels.

Thus, there is a need for an improved composition for alleviating conditions, e.g. pain, irritation and/or itching, associated with vaginal dryness.

### SUMMARY

Consequently, the present invention seeks to mitigate, alleviate, eliminate or circumvent one or more of the above-identified potential deficiencies in the art and disadvantages singly or in any combination by providing a composition composition comprising:
a thickener;
a non-ionic, water-soluble poly(ethylene oxide) polymer having molecular weight of at least 100,000 Da, such as at least 500,000, at least 1,000,000 Da, or at least 5,000,000 Da; and
at least 95 wt % water. The composition is used for alleviating conditions associated with vaginal dryness. Such conditions include pain, irritation, inflammation, and/or itching in the vaginal introitus and/or the vagina. Further, the composition may be used for de-sensitizing the vaginal introitus and/or the vagina.

A further aspect of the invention relates a kit for providing a composition which may be used to alleviate conditions associated with vaginal dryness. The kit comprises a first aqueous mixture comprising a non-ionic, water-soluble poly(ethylene oxide) polymer having molecular weight of at least 100,000 Da, and at least 95 wt % water, and a second aqueous mixture comprising a thickener and at least 95 wt % water. The composition is obtained by blending the first and the second mixture.

Further advantageous features of the invention are defined in the dependent claims. In addition, advantageous features of the invention are elaborated in embodiments disclosed herein.

### DETAILED DESCRIPTION OF PREFERRED EMBODIMENTS

The present inventor has previously disclosed that aqueous compositions, comprising at least one thickener and at least one compound forming a shear-thickening fluid when mixed with water, which composition comprises at least 95 wt % water, are useful as sexual lubricants (cf. WO 2011/064383). Such compositions were found to be more lubricating than conventional sexual lubricants. As such compositions may be provided without bio-degradable components, whereby the need for preservatives may be dispensed with, they are suitable for administration to the vagina providing essentially no effect on the bacterial flora in the vagina. Further, GB2459732 discloses personal lubricants for lubricating the skin during sexual activity, which can be frozen and melts during use to provide lubrication.

While WO 2011/064383 relates to the lubricating properties of such compositions, no medical usage, such as pain relief, of such compositions has previously been disclosed.

It has now surprisingly been found that compositions comprising a thickener; a non-ionic, water-soluble poly(ethylene oxide) polymer having molecular weight of at least 100,000 Da; and at least 95 wt % water, except for its lubricating properties also has protecting properties. Especially, it was found that such compositions are more effective than oil, silicone or water based gels of the art in alleviating conditions associated with vaginal dryness. Thus, pain, irritation and itching in the vaginal introitus and the lower vagina, often experienced in women suffering from decreased vaginal lubrication and/or vaginal dryness, may be alleviated by use of such a composition.

Thus, an embodiment relates to a composition comprising a thickener; a non-ionic, water-soluble poly(ethylene oxide) polymer having molecular weight of at least 100,000 Da; and at least 95 wt % water for alleviating conditions associated with vaginal dryness. Conditions associated with vaginal dryness include pain, irritation, inflammation and/or itching in the vaginal introitus and/or the vagina. According to an embodiment, the condition is a therapeutic condition. According to an embodiment, the condition to be alleviated is pain. As explained above, while conventional lubricants may provide at some temporarily alleviation of irritation and itching, they provide essentially no pain soothing effect. The finding that the present composition, not only provides lubricating effect, but importantly also protecting and de-sensitizing properties implies that relief of pain associated with vaginal dryness may be provided. According to another embodiment, the condition to be alleviated is irritation and/or inflammation. According to yet another embodiment, the condition to be alleviated is itching. Vaginal dryness may have various causes. A common cause is decreased estrogen production. As an example the present composition may be thus used to alleviate conditions associated with vaginal dryness in women during or post the menopause. Similarly the present composition may be used to alleviate conditions associated with vaginal dryness in women treated with anti-estrogenic medication. The present composition may also be used to alleviate conditions associated with vaginal dryness, in pregnant women and women breast-feeding children.

Except for anti-estrogenic medication, also other medications, such as anti-histamines, medications with anti-cholinergic or sympathomimetic effects, drugs for allergic, cardiovascular, psychiatric conditions, oral contraceptives and contraceptive creams and foams may also decrease or inhibit the inherent vaginal lubrication. Accordingly, the present composition may be used to alleviate conditions associated with vaginal dryness caused by such medications as well.

Furthermore, the composition was surprisingly found to also alleviate conditions associated vaginism or vestibulitis. Especially, the composition may be used to alleviate the pain experienced by women suffering from vaginism or vestibulitis. As conventional lubricants provide essential no symptom relief for women suffering, it is believed that properties of present composition are related to is protecting and de-sensitizing properties.

Without being bond to any theory, it is believed that the composition, except for its lubricating properties, also has protecting properties, whereby being especially useful in treating pain in subjects suffering from vaginism or vestibulitis. According to an embodiment, the composition is for symptomatic treatment of vaginism and/or vestibulitis. As the composition has protecting properties it may, except for alleviating pain associated with vaginal dryness, also be used to alleviate irritation and/or inflammation due to vaginal dryness.

It seems that the composition exerts said protecting effect by providing a temporary, non-moving, solid film protecting the vaginal mucosa, once exposed to shearing strains, such as by relative movement of the opposing walls of the vagina. Without being bond to this theory, it seems that the presumed presence of such a temporary film implies that the labile vaginal mucosa only is exposed to compressing strains, as the shear strains are disengaged by the film.

A simple experiment, wherein the present composition was applied to the one of the two feet soles of a subject, did support the formation of such a temporary, non-moving solid film, as the subject did not react to tickling at the treated sole. The present composition may thus also be used to de-sensitize the vaginal introitus and/or the vagina.

In contrast conventional gels provide passive lubricating effect, reducing but not disengaging shear strains. Further, the lubricating effect of conventional gels, such as aqueous gels comprising swollen polysaccharides, will rapidly decrease once the water content gradually is absorbed or evaporates, leaving a sticky residue, which has to be washed away. The subsequent washing may even worsen the dryness, providing a vicious circle.

The thickener should preferably be pharmaceutically acceptable one. Pharmaceutically acceptable is intended to mean that the compound, at the dosage and concentrations employed, does not cause any unwanted effects. Preferably, pharmaceutically acceptable compounds are compounds approved for use as pharmaceutical excipients, such as excipients approved for use pharmaceuticals for topical administration to the skin.

Thickeners are typically compounds increasing the viscosity of liquids to which they are added, and thereby thickening such solutions. Without the presence of a thickener the composition will not be viscous enough for controlled use.

Preferably, the thickener may be a compound forming a shear-thinning fluid when mixed with water. By having a thickener forming a shear-thinning fluid when mixed with water, rather than any thickening agent, the lubricious aqueous composition will more easily spread to a thin layer when applied to the vagina.

A composition comprising a thickening agent not forming a shear-thinning fluid when mixed with water may, for some type of applications, be too gel-like, or viscous, to be efficiently spread.

Furthermore, the thickener may preferably be either water-soluble or a compound which swells in the presence of water to form a gel. The dissolution or swelling may require the addition of a base or an acid in order to adjust the pH of the solution or dispersion to be formed. Most preferably, the thickener is a water insoluble hydrophilic compound, which swells in the presence of water to form a gel.

As stated above it is preferred if the thickener is pharmaceutically acceptable. In relation to this, it is preferred that the thickener has such a high molecular weight that it can not penetrate human skin. Potential side effects, local as well as systemic, are minimized if the thickener never enters the body.

One example of a preferred group of thickeners to be used in such a composition, as disclosed herein, is polymers of crosslinked polyacrylate, such as high weight polymers of crosslinked polyacrylate.

Crosslinked polyacrylates are in principle non-degradable by bacteria. Thus, they will not promote growth of bacteria in the vagina or disturb the natural bacterial flora. According to an embodiment, non-degradable by bacteria, i.e. non-biodegradable, is intended to mean that the bacteria are not able to degrade the compound or polymer. Thus, the compound or polymer does not serve as a nutrient.

By using low amounts of non-biodegradable thickeners, such as polymers of crosslinked polyacrylate, as thickener, the need to add preservatives may be dispensed with.

An example of high weight polymers of crosslinked polyacrylate, are Carpopol® polymers. One especially preferred Carbopol® polymer is Carbopol® Ultrez 21 (INCI name: Acrylates/C10-30 alkyl acrylate crosspolymer), which may obtained from Lubrizol Advanced Materials Europe BVBA in Belgium.

Crosslinked polyacrylates are an example of a group of thickeners requiring the addition of a base, such as sodium hydroxide, to swell in water. It seems as if the protonated polymer does not readily swells in water, whereas the de-protonated crosslinked polyacrylate does.

According to one embodiment, high weight polymers of crosslinked polyacrylate are intended to mean polymers, wherein the individual crosslinked polyacrylate polymer chains have an average molecular weight of at least 100,000, such as at least 250,000, as measured by gel permeation chromatography using linear polyacrylic acid as reference, if the individual polyacrylate polymer chains have been polymerized under the same conditions as the crosslinked polymer, and using the same recipe as the crosslinked grades, but without any crosslinking monomer.

The amount of the thickener in the composition, as disclosed herein, may be equal to or less than 2.5 wt %, such as equal to or less than 1.0, 0.5, 0.1, 0.05, or 0.01 wt %. As the thickener, in contrast to water, will not evaporate, a low content of thickener is preferred. However, the content has to be high enough to noticeably affect the viscosity of the composition and give the desired effects described above. Accordingly, the lubricious aqueous composition may comprise at least 0.005 wt % of the thickener, such as at least 0.01 or 0.1 wt %.

The water content of the aqueous composition is at least 95 wt. %. Further, in some embodiments the water content is at least 98 wt %, or even at least 99.0 or at least 99.5 wt %. The high water content provides a significant advantage, as very limited amounts of exogenous substances are administrated to the vagina, compared to water, oil, or silicon based gels of the art, when the composition is being used to alleviate conditions associated with vaginal dryness.

Without being bound to any theory, it is believed that the property of the composition, making it useful for alleviating conditions associated with vaginal dryness, is not only related to its previously disclosed lubricating properties, but also to its protecting properties. As already described, it seems that the composition exerts said protecting effect by providing a temporary, non-moving, solid film protecting the vaginal mucosa, once exposed to shearing strains, such as by relative movement of the opposing walls of the vagina. As each wall will be covered by a non-moving film, the labile mucosa will only be exposed to compressing strains, as the shear strains are disengaged by the opposing films. The presumed presence of such a temporary, non-moving, solid film may be explained by the non-Newtonian properties, i.e. shear-thickening properties, provided by the poly(ethylene oxide) polymer. As already mentioned, such a film effectively protects sensitive mucosa of the vagina, whereby pain, irritation, inflammation, and itching, associated with vaginal dryness, may be alleviated.

Non-ionic, high molecular weight, water-soluble poly(ethylene oxide) polymers (PEO-polymers) having a molecular weight of at least 100,000 Da are sold by the Dow Chemical Company under the trademark PolyOx. A preferred type of PolyOx is PolyOx WSR 301.

According to an embodiment, the non-ionic, high molecular weight, water-soluble poly(ethylene oxide) polymer has molecular weight of at least 500,000, at least 1,000,000 Da, or even at least 5,000,000.

While poly(ethylene oxide) polymer is a preferred component for providing the composition with protecting properties, it may, according to an embodiment, be replaced with a compound forming a shear-thickening fluid when mixed with water. Such a compound may be a water soluble polymer. Further, such a polymer does typically have a high molecular weight, such as a molecular weight of at least 100,000 Dalton (Da), such as at least 500,000, at least 1,000,000 Da or even at least 5,000,000 Da. In addition, such a polymer is typically a linear, i.e. non-branched, polymer.

Similar to cross-linked polyacrylate, high molecular weight, non-ionic, water-soluble poly(ethylene oxide) polymers are in principle non-degradable by bacteria. Thus, they will not promote growth of bacteria in the vagina or disturb the natural bacterial flora. By using low amounts of non-biodegradable compound forming a shear-thickening fluid when mixed with water, such as water-soluble poly(ethylene oxide) polymers, the need to add preservatives may be dispensed with. Further, the composition may not promote bacterial growth to any extent.

The amount of the non-ionic, water-soluble poly(ethylene oxide) polymer, in the composition as disclosed herein may be equal to or less than 2.5 wt %, such as equal to or less than 1.0, 0.5, 0.1, 0.05, or 0.01 wt %. As the non-ionic, water-soluble poly(ethylene oxide) polymer, in contrast to water, will not evaporate, a low content of the non-ionic, water-soluble poly(ethylene oxide) polymer is preferred, so that a minimum of compound is left if/when the water of the composition has evaporated. However, the content has to be high enough to noticeably affect the viscosity of the composition. Accordingly, the composition may comprise at least 0.005 wt % of the non-ionic, water-soluble poly(ethylene oxide) polymer, such as at least 0.01 or 0.1 wt %.

The pH of the composition should preferably be slightly acidic, i.e. below 7.0. An acidic pH will enhance the shelf-life of the product. Furthermore, the rheological properties, especially if a crosslinked polyacrylate is used as thickener, may depend on the pH of the composition. An aqueous solution comprising cross-linked polyacrylates may further be opalescent at a low pH. Accordingly, it is preferred if the pH of the composition is between 3 and 5, such as between 3 and 4 or between 4 and 5, e.g. between 4.7 and 4.9.

As indicated above the pH may affect the rheological properties of a lubricant comprising crosslinked polyacrylate as thickener. An increase in pH will increase the viscosity of the lubricant.

In order to obtain a pH of 3 to 5, it may be necessary to adjust the pH of the lubricious aqueous composition by adding an acid or a base. Preferably, sodium hydroxide is used to adjust the pH. Sodium is a suitable counter-ion and hydroxide will give water when neutralized.

As the composition is to be applied to the vagina, a pH close the natural pH of the vagina is preferred. The normal vaginal pH is 3.8 to 4.5. Thus, the pH of the lubricious aqueous composition according to an embodiment is 3.8 to 4.5.

Another preferred embodiment relates to an aqueous composition, for alleviating conditions associated with vaginal dryness, essentially consisting of 98 to 99.9 wt % water, 0.05 to 1 wt % of an essentially none bio-degradable thickener, such as a high weight polymer of crosslinked polyacrylate, eg. Carbopol® Ultrez 21, and 0.05 to 1 wt % of non-ionic, water-soluble poly(ethylene oxide) polymer having a molecular weight of at least 100,000 Da, e.g. PolyOx WSR 301. Such a composition may have a pH of 3.8 to 4.5. Further, the water used to obtain the composition may have a total organic carbon content (TOC) of 20 μg/L or less, such as 5 μg/L or less. As such composition comprises only essentially non-biodegradable components, it will not promote growth of micro-organisms. In addition, the absence of any preservative will minimize the affect on the natural urogenital flora of the woman. Further, as its pH corresponds to the normal pH of the human vagina and as it does not comprise any preservatives, it will not interfere with or affect the natural urogenital flora of the woman.

According to an embodiment, the composition essentially consists of essentially non-biodegradable components and water, thus not promoting growth of microorganisms.

As denoted above, the need to add preservatives may be dispensed with if the lubricious aqueous composition only comprises components being essential non-biodegradable. As water may contain small amount of organic compounds, which may promote growth of microorganisms, the water used to produce the lubricious aqueous composition may be purified water. Examples of purified water includes distilled water, water purified by reverse osmosis. According to an embodiment, the water used to obtain the lubricious aqueous composition has a total organic carbon (TOC) content of 20 μg/L or less, such as 5 μg/L or less.

The Total Organic Carbon (TOC) is commonly defined as any compound containing a carbon atom except for CO₂, and related substances such as carbonate, bicarbonate and the like. As carbonates are considered to be fully oxidized, they do not form part of the constituents of TOC. Considering this exception to the definition of TOC, an alternative definition of TOC might be total oxidizable carbon.

According to embodiment, TOC, as used herein, is to be determined in accordance with the method 415.3 disclosed in “DETERMINATION OF TOTAL ORGANIC CARBON AND SPECIFIC UV ABSORBANCE AT 254 nm IN SOURCE WATER AND DRINKING WATER” (EPA Document #: EPA/600/R-05/055) issued by National Exposure Research Laboratory Office of Research and Development U.S. Environmental Protection Agency (EPA) Cincinnati, Ohio.

According to another embodiment, the composition is provided as a kit comprising two mixtures. By blending the two mixtures a composition, as described herein, is obtained. The mixtures are typically blended prior to use, such as less than one week before use. Except for the two mixtures, the kit may comprise a container for the composition once obtained, such as a tube or flask. The first mixture is an aqueous mixture comprising a non-ionic, water-soluble poly(ethylene oxide) polymer having molecular weight of at least 100,000 Da, such as at least 500,000, at least 1,000,000 Da, or at least 5,000,000 Da, and at least 95 wt % water. The second mixture is also an aqueous mixture comprising at least 95 wt % water. Further, the second mixture comprises a thickener of the type described herein. It has been seen that the properties of the present composition are somewhat reduced over time. Thus, it may be advantageous to provide a kit allowing for preparation of the composition shortly before use, whereby prolonged shelf life may be provided.

The role of each of components obtained by mixing the mixtures has already been described herein. The same guiding principles as applies for providing the composition applies also for the provision of the two mixtures.

Especially, as already described the thickener may be cross-linked polyacrylate. Further, the first aqueous mixture and the second aqueous mixture may each comprise at least 98 wt % water, such as at least 99.0, or at least 99.5 wt % water. The second aqueous mixture may comprise between 0.01 and 1 wt % of the thickener. Further, the first aqueous mixture may comprises 0.01 and 1 wt % of the water-soluble poly(ethylene oxide) polymer having molecular weight of at least 100,000 Da. The pH of the each of the two aqueous mixtures may be between 3 and 5, such as between 3 and 4, or between 4 and 5. Further, the second aqueous mixture may essentially consists of 98 to 99.9 wt % water and 0.05 to 1 wt % of the thickener. Similarly the first aqueous mixture may essentially consists of 98 to 99.9 wt % water and 0.05 to 1 wt % of the water-soluble poly(ethylene oxide) polymer having molecular weight of at least 100,000 Da. Also, the first aqueous mixture and the second aqueous mixture may each essentially consists of essentially non-biodegradable components and water. According another embodiment, the composition described herein is obtained by mixing a first aqueous mixture comprising a non-ionic, water-soluble poly(ethylene oxide) polymer having molecular weight of at least 100,000 Da, such as at least 500,000, at least 1,000,000 Da, or at least 5,000,000 Da, and at least 95 wt % water, and a second aqueous mixture comprising a thickener and at least 95 wt % water prior to use of the composition. The thickener is of the type described herein.

While the present composition has been described as being used to alleviating conditions associated with vaginal dryness, it may also be used in the manufacture of a product for the alleviation of conditions associated with vaginal dryness. Further, a method for alleviating conditions associated with vaginal dryness may include the administration of the composition to a woman suffering from conditions associated with vaginal dryness.

Without further elaboration, it is believed that one skilled in the art can, using the preceding description, utilize the present invention to its fullest extent. The preferred specific embodiments described herein are, therefore, to be construed as merely illustrative and not limitative of the remainder of the description in any way whatsoever. Further, although the present invention has been described above with reference to specific embodiments, it is not intended to be limited to the specific form set forth herein. Rather, the invention is limited only by the accompanying claims and, other embodiments than the specific above are equally possible within the scope of these appended claims, e.g. different than those described above.

In the claims, the term “comprises/comprising” does not exclude the presence of other elements or steps. Additionally, although individual features may be included in different claims, these may possibly advantageously be combined, and the inclusion in different claims does not imply that a combination of features is not feasible and/or advantageous.

In addition, singular references do not exclude a plurality. The terms “a”, “an”, “first”, “second” etc do not preclude a plurality.

### EXPERIMENTAL

The following examples are mere examples and should by no mean be interpreted to limit the scope of the invention. Rather, the invention is limited only by the accompanying claims.

An aqueous composition comprising cross-linked polyacrylate and poly(ethylene oxide) polymer (cf. WO 2011/064383) was provided as described below in the form of a gel. The gel was evaluated in a pilot study, including ten subjects, to verify its effect in alleviating symptoms associated with vaginal dryness. Further, a complementary pilot study has been performed.

Water

Water used in preparing the mixtures 1 to 3 was obtained by de-ionizing tap water followed by filtration through active carbon or radiation with UV-light.

Mixture 1

Water (200 kg) was heated to 100° C. and stirred to form a vortex. PolyOx WSR 301 powder (1.2 kg; The Dow Chemical Company) was sifted into the heated water and stirred heavily until a clear solution was obtained.

Mixture 2

Water (50 kg) was heated to boiling. Once the temperature had fallen to 40° C., Carbopol Ultrez 21 powder (500 g Lubrizol Advanced Materials Europe BVBA) was sifted into the water. The mixture was left until the Carbopol Ultrez 21 powder has swollen.

Mixture 3

Water (50 kg) was heated to boiling and aqueous (20%) sodium hydroxide (250 g) was added.

Gel

Mixture 1, having a temperature of approx. 20° C., was gently stirred and mixture 2 was added thereto, whereafter mixture 3 was added to obtain the gel. The gel was stored under oxygen free atmosphere.

### Example 1

### Pilot Study

Nine patients who had made an appointment at a gynecologist for conditions associated with vaginal dryness and one control patient were included in a pilot study for evaluating the medical effects of the composition. The ten patients in the study stated various reasons for consulting a gynecologist (cf. table 1) at a questionnaire. Each patient was examined and after receiving informed consent with written information (1), a tube of gel with no information on it and a questionnaire to fill at home (2) was handed out to the patients.

They were seen, after ad hoc self-medication, and after about one month.

Five patients reported itching as dominating symptom; four of them reported the itching to inhibit their sex-life. One patient reported itching combined with overactive bladder (OAB), one experienced itching as an effect of anti-estrogen treatment, one patient had vaginism/painful intercourse, while one patient only wanted to increase lust during intercourse. Three patients reported vaginal dryness during and after anti-estrogen treatment.

One patient with an obvious lichen sclerosis et atrophicus vulvae, a disease where one not expects any effect of the gel, was asked about participation and information was given exactly as above to verify a possible placebo effect from the information.

At the second appointment—after about one month—the questionnaire was returned.

Results

All ten patients except one—the control patient with lichen sclerosis and athrophicus vulvae—reported improvement with much less problems (cf. Tab.1). The patients had to “fully agrees” (5 points), “partly agrees” (3 point)”, and does not agree at all” (1 point) with a couple of claims. Five (5) women had used the gel daily, while another five (5) patients had used the gel ad hoc.

Four (4) patients fully agreed that they had had a very satisfactory help from the gel, while four (4) agreed that the help was satisfactory. No side effects were noted. Nine (9) patients stated that they would recommend the gel to a female friend. The only woman not to agree was the “control patient”.

Opportunities for “free comments” were given, and used by all patients. The comments are referred below;

1/ Comfortable, better than conventional gels

2/ No itching any more.

3/ Orgasm with the gel; almost never without.

4/ I had problems with less sex drive, now much better.

5/ The gel was thin-flowing, though comfortable. Almost cured from Protopic. (Controlpat.)

6/ Very satisfied.

7/ Much better than earlier preparations (conventional gels).

8/ Great, Excellent,/anti estrogen treatment/

9/ Very helpful though not penetration.

10/ More comfortable than other preparations.

Comments

The patients significantly emphasize a better sex-life, also if the preliminary problem was dryness, itching and/or burning pain. The need for an efficient, non-hormonal gel is apparent for patients on anti-estrogen therapy. All patients reported positive effects, including the lichen patient (no. 5) who was not cured. The woman with “vaginism” could be digitally examined without pain with the gel. She has restarted her sex-life though not yet having had penetration sex.

TABLE 1
Summary of the pilot study

| | | | | | | | Administration | | | Evaluation | | | |
|---|---|---|---|---|---|---|---|---|---|---|---|---|---|
| | | Diagnosis by | Conditions stated by patient | | | | Once | >Once | Ad | Very | | Will | Side |
| Pat. | Age | physician | Pain | Itching | Dryness | Dyspareunia | daily | daily | Hoc | satisfactory | Helped | recommend | effects |
| 1 | 45 | OAB | X | | X | | X | | | 2 | 4 | 5 | no |
| 2 | 69 | Itching | | X | X | | | X | | 5 | | 5 | no |
| 3 | 68 | Dyspareunia | | | X | X | | | X | 4 | 1 | 3 | no |
| 4 | 44 | Fading libido | X | | X | X | | | X | 4 | 4 | 4 | no |
| 5 | 52 | Lichen | X | | X | | | X | | 3 | 4 | 2 | no |
| | | (control) | | | | | | | | | | | |
| 6 | 57 | Dryness | | | X | X | | | X | 5 | 1 | 4 | no |
| 7 | 59 | Dryness | X | | X | X | | | X | 4 | 1 | 4 | no |
| 8 | 62 | Adenoca uteri | | | X | | X | | | 5 | | 5 | no |
| 9 | 44 | Vaginism | X | X | X | | X | | | 4 | | 4 | no |
| 10 | 58 | Dyspareunia | | | X | | | | X | 5 | 5 | 5 | no |

### Example 2

### Expanded Pilot Study

The expressed purpose of the studies was to test the effect of the Gel on dyspareunia and/or vaginism. Other intentions were to study the gel as a hormone free moistening gel in patients with symptoms from inadequate vaginal lubrication. The patients in the studies were recruited and examined at two different sites, in the Malmoe area and the Gothenburg area (both in Sweden). Recruitment and examination was performed by two gynecologists, one at each site, using the same questionnaire and the same inclusion criteria. Any type of infection excluded the patient from the study, while inflammatory reaction of unknown reason or epithelial atrophy was accepted. The patients were seen, after about one month of treatment. The two studies differ somewhat in structure; the presentation of the Malmoe material is based on a diagnosis, resulting from one or several symptoms i.e. dryness, itching and/or dyspareunia. The Gothenburg material is based on the recruitment only the dominant one of these symptoms.

After informed consent with written information (1), one tube of gel with no information on it and a questionnaire to fill in at home (2) was handed to 20 patients in the Malmoe area and 21 patients in the Gothenburg area. 18 respectively 20 questionnaires were returned and summarized below.

### Results from the Malmoe Study

### Expansion of the Study in Example 1

N=18. The mean age was 59.06 years SD 7.79

In 16/18 patients in the Malmoe area reported burning, itching and/or dryness as a dominant symptom; seven of them reported the itching to inhibit their sex-life. One patient reported itching combined with OAB, three experienced itching as an effect of anti estrogen treatment one patient had vaginism/painful intercourse while one patient only wanted to increase lust during intercourse. One patient with an obvious lichen sclerosis et atrophicus vulvae, a disease where you might not expect any effect of the gel, was asked about participation and information was given exactly as above to verify a possible placebo effect from the information.

All 18 patients except one—the control patient with lichen sclerosis and atrophicus vulvae—reported improvement with much less problems. The patients had to “fully agree” (5 points) till “does not agree at all” (1 point) with a couple of claims. Seven women had used the gel daily, while thirteen patients had used the gel also ad hoc. Five patients had chosen to use the gel>one time daily, Eight patients fully agreed that they had had a very satisfactory help from the gel, seven of them complaining of dyspareunia, while another eight—six of them with dyspareunia—agreed that the help was satisfactory. 12 patients agreed that they would recommend the gel to a girl friend, while only two patients—one of them the “control patient” with lichen—disagreed on effect. One patient with vaginism had not experienced sexual penetration but accepted a new vaginal examination with the gel.

15/18 patients experienced dyspareunia as part of their diagnosis 7/15 reported excellent help and another 6 reported good help from the gel. One patient experienced acceptable help while another patient remarked that she found the gel to dry faster than other products. No side effects were noted.

Opportunities for “free comments” were given, and used by all patients. The comments are referred to below;
1/ Comfortable, better than “XX” Smoothing Cream OAB+Cystitis
2/ No itching any more. Great Thanks/Name/Itching
3/ Orgasm with the gel. Almost never without. Dyspareunia
4/ I had problems with less sex-drive, now much better. Fading Libido
5/ Thin, though comfortable. Almost cured from Protopic. Lichen sclerosis/Controlpatient/
6/ Very satisfied.Dryness
7/ Much better than earlier preparations. Dryness
8/ Great, Excellent. Adenoca. uteri
9/ Very helpful though not penetration at sex Vaginism
10/ More comfortable than other preparations. Dyspareunia
11/ Better applicator Dyspareunia
12/ Anti-estrogenic treatment Very satisfied/Ca.mammae/Dyspareunia
13/ Started local estrogen simultaneously (!!!) Dyspareunia
14/ No problems what so ever after two weeks of daily treatment. Dyspareunia
15/ Sooner dry than with other products. Dyspareunia
16/ Great!! Excellent!!/Ca.mammae/Dyspareunia
17/ Very satisfied Dyspareunia
18/ No comment—questionnaire sent as letter/Dyspareunia

Results from the Gothenburg Study

N=20. The mean age was 59.8 years SD 17.39.

Three patients reported itching, another four patients reported burning sensations, while 10 patients with dyspareunia and/or vaginism tried the gel. The three patients with itching reported good (2), or very good (1) relief. The results in the patients with burning sensations were; one very good two good and one “sometimes”. Out of the 10 patients with dyspareunia/vaginism, eight reported very good or god results. Only one patient reported no effect. 10 out of 19 (answering) patients reported that they would recommend the gel. Also in this material a “control patient” applying for urethritis was included. She reported a “cooling” effect on the burning sensations but no effect on her urethritis.

Also in the Gothenburg Study opportunities for “free comments” were given and answered by 18/20 patients:

1/ Not very different from XX-crëme Dryness

2/ Feels comfortable and removes the dryness Itching

3/ Cooling but no effect on the urethritis Urethritis/dryness/itching

4/ Burning

5/ Removed dyspareunia Dyspareunia

6/ Would wish thicker consistency to use easier Vaginism

7/ Very satisfied. Dyspareunia

8/ Did not relieve the burning feeling. Too thin consistency Burning

9/ Excellent effect against dryness Dryness

10/ No report what so ever No report

11/ /No comment/Itching

12/ Too thin consistency Burning

13/ Excellent against dyspareunia Dyspareunia

14/ Nice to apply Itching

15/ Has been excellent for me Dyspareunia

16/ I think it is excellent and helps Dyspareunia

17/ Advantage of no odor. Thin consistency—which I like Dyspareunia

18/ Discolored/brown/discharge but helped against vaginism. Vaginism

19/ Helped my sex-life—no pains Dyspareunia

20/ Excellent Dyspareunia

21/ Almost always excellent. Burning

Comments

In the total material 25/38 patients complained about dyspareunia. 10/25 patients reported an excellent help, 12/25 reported good help 2/25 pat were helped while only 1/25 patients reported no change.

The patients emphasized a better sex-life, also if the preliminary problem was dryness and/or burning pain. Thus more than 50% of patients with dryness, itching and or burning as apart of dyspareunia (8/14) reported excellent results while additional four patients reported very good relief.

The need for an efficient and non-hormonal gel is apparent for patients on anti estrogen therapy. Two patients operated on for estrogen dependent tumors, one of them on anti-estrogen medication, reported excellent results.

All patients, except two—one in each material—reported positive effect. Both studies include one “control patient” to show that the presentation of the gel is of low importance. Thus also the “control”—patients reported some effect—though they were not cured. The woman with “vaginism” could be digitally examined without pain with the gel. She has restarted her sex-life though not with penetration.

### CONCLUSION

It is obvious that the gel has great and advantageous effect in women with “trophic” problems. It is even more interesting that the gel not only has lubricating effect, but also a protective effect. The gel thus has properties, except for its lubricating ones, making it useful for medical applications.

1. A method of alleviating conditions associated with vaginal dryness, or for d-sensitizing the vaginal introitus and/or the vagina, said method comprising administering a composition comprising:
a thickener;
a non-ionic, water-soluble poly(ethylene oxide) polymer having molecular weight of at least 100,000 Da; and
at least 95 wt % water, to the vagina or the vaginal introitus of woman in need thereof.

2. The method according to claim 1, wherein said condition associated with vaginal dryness is one or several of the following: pain, irritation, inflammation and/or itching in the vaginal introitus and/or the vagina or wherein the vaginal introitus and/or the vagina is de-sensitized to alleviate pain, irritation, inflammation itching.

3. The method according to claim 2, wherein said condition associated with vaginal dryness is pain in the vaginal introitus or the vagina, or wherein the vaginal introitus and/or the vagina is de-sensitized to alleviate pain.

4. The method according to claim 1, wherein said thickener is cross-linked polyacrylate.

5. The method according to claim 4, wherein the composition comprises at least 98 wt % water.

6. The method according to claim 5, wherein the composition comprises between 0.01 and 1 wt % cross-linked polyacrylate, and 0.01 and 1 wt % of the water-soluble poly(ethylene oxide) polymer having molecular weight of at least 100,000 Da.

7. The method according to claim 6, wherein the pH of the composition is between 3 and 5.

8. The method according to claim 7, wherein said composition essentially consists of 98 to 99.9 wt % water, 0.05 to 1 wt % cross-linked polyacrylate, and 0.05 to 1 wt % of the water-soluble poly(ethylene oxide) polymer having molecular weight of at least 100,000 Da.

9. The method according to claim 1, wherein said composition essentially consists of essentially non-biodegradable components and water.

10. The method according to claim 1, wherein said conditions associated with vaginal dryness is due to decreased estrogen production.

11. The method according to claim 10, wherein said woman is in or post the menopause, treated with anti-estrogenic medication, pregnant and/or breast-feeding children.

12. The method according to claim 1, wherein conditions associated with vaginal dryness is due to medication.

13. The method according to claim 1, wherein said woman suffers from vaginism or vestibulitis.

14. The method according to claim 1, wherein the composition is obtained by mixing a first aqueous mixture comprising a non-ionic, water-soluble poly(ethylene oxide) polymer having molecular weight of at least 100,000 Da and at least 95 wt % water, and a second aqueous mixture comprising a thickener and at least 95 wt % water less than one week before administering the composition to the woman.

15. A kit for providing a composition for use in the method according to claim 14, said kit comprising a first aqueous mixture comprising a non-ionic, water-soluble poly(ethylene oxide) polymer having molecular weight of at least 100,000 Da, and at least 95 wt % water, and a second aqueous mixture comprising a thickener and at least 95 wt % water.

16.-18. (canceled)

19. The kit according to claim 15, wherein said thickener is cross-linked polyacrylate.

20. The kit according to claim 15, wherein the first aqueous mixture and the second aqueous mixture each comprises at least 98 wt % water.

21.-22. (canceled)

23. The kit according to claim 15, wherein said second aqueous mixture essentially consists of 98 to 99.9 wt % water and 0.05 to 1 wt % crosslinked polyacrylate, and wherein said first aqueous mixture essentially consists of 98 to 99.9 wt % water and 0.05 to 1 wt % of the water-soluble poly(ethylene oxide) polymer having molecular weight of at least 100,000 Da.

24.-28. (canceled)

29. The method according to claim 12, wherein said medication is selected from the group consisting of anti-histamines, medications with anti-cholinergic or sympathomimetic effects, drugs for allergic, cardiovascular, and psychiatric conditions, oral contraceptives, and contraceptive creams and foams.
